(19) United States
(12) Patent Application Publication
(10) Pub. No.: US 2009/0298068 A1
(43) Pub. Date: Dec. 3, 2009

(54) METHOD AND TEST KIT FOR THE DIAGNOSIS AND/OR MAKING PREDICTIONS ABOUT AND/OR FOR THE ASSESSMENT OF THE EFFICACY OF THERAPEUTIC AGENTS FOR THE TREATMENT OF OVARIAN CANCER AND METHOD OF PLANNING A REGIMEN FOR THE TREATMENT OF OVARIAN CANCER

(76) Inventors: Michael Krainer, Klagenfurt (AT); Dietmar Pils, Wien (AT); Robert Zeillinger, Blumau-Neurisshof (AT)

Correspondence Address: K.F. ROSS P.C., 5683 RIVERDALE AVENUE, SUITE 203 BOX 900, BRONX, NY, 10471-0900, US

(21) Appl. No.: 12/293,275

(22) PCT Filed: Mar. 16, 2007
(86) PCT No.: PCT/EP2007/002338

§ 371 Date: Jan. 31, 2009

(30) Foreign Application Priority Data
Mar. 16, 2006 (AT) A441/2006

(51) Int. Cl. C12Q 1/68

(52) U.S. Cl. 435/6

(57) ABSTRACT

The invention relates to a method and a test kit for diagnosing ovarian cancer and/or making predictions in case of ovarian cancer as well as a method for estimating the effectiveness of therapeutic agents during the treatment of ovarian cancer, the promoter hypermethylation of the TUSC3 marker in a biological sample that is to be analyzed, preferably in a tissue sample or biological liquid that is to be analyzed, being measured. The result of said method can be used for planning an ovarian cancer treatment.

The invention relates to a method and a test kit for the diagnosis of ovarian cancer, and/or for making predictions about ovarian cancer, and to a method of the assessment of the efficacy of therapeutic agents for the treatment of ovarian cancer, and to a method of planning a regimen for the treatment of ovarian cancer.

Malignant ovarian cancer is the most lethal gynecological cancer disease and at 6% the fourth most frequent cause of death among women with cancer in Western countries. According to estimates, one of 70 women develops ovarian cancer during her life time, with an average survival rate of 4.5 years. More recent cancer statistics estimate 22,200 new cases and 16,210 fatalities per year in the United States (Jemal A., Murray T., Ward E., Samuels A. Cancer Statistics, 2005 (vol. 55, pg. 10, 2005). Ca-A Cancer Journal for Clinicians 2005; 55(4):259).

A total of 5% to 10% of all ovarian cancer patients have a hereditary predisposition and primarily suffer from the so-called “Hereditary breast and ovarian cancer syndrome” or the “Lynch II syndrome”. Most cases, however, are sporadic instances of ovarian cancer. For genetically-associated high-risk patients, prophylactic ovariectomy reduces the risk and intensified screening allows earlier diagnoses for the future. In the general population, early diagnosis is the primary task, since more than three quarters of cases are diagnosed at a later stage (FIGO stages III and IV). All these patients require a multimodal therapy, comprising optimized debulking surgery and systemic chemotherapy.

Molecular markers that correlate with the start of treatment, and therefore support treatment decisions, are required.

Presently recognized prognostic markers comprise the initial staging, grading and size of the remaining, non-removable tumor mass following cytoreductive surgery (van der Burg M E. Advanced ovarian cancer. Curr Treat Options Oncol 2001; 2(2): 109-118; Holschneider C H, Berek J S. Ovarian cancer: Epidemiology, biology and prognostic factors. Seminars in Surgical Oncology 2000; 19(1):3-10). In addition to the tumor marker “Cancer Antigen 125” (CA-125) (Makar A P, Kristensen G B, Kaern J, Bormer O P, Abeler V M, Trope C G. Prognostic value of pre- and postoperative serum CA 125 levels in ovarian cancer: new aspects and multivariate analysis. Obstet Gynecol 1992; 79(6): 1002-1010), molecular markers for the early detection and prognosis do not exist in clinical practice (Ludwig J A, Weinstein J N. Biomarkers in Cancer Staging, Prognosis and Treatment Selection. Nat Rev Cancer 2005).

The aim of the present invention is the diagnosis and making predictions about ovarian cancer, the creation of a diagnostic and prognostication method of ovarian cancer and a test kit therefor, and the creation of a method of the assessment of the efficacy of therapeutic agents for the treatment of ovarian cancer and a method of planning a regimen for the treatment of ovarian cancer.

The inventors of the present invention have found that TUSC3 is suited as a molecular marker for ovarian cancer. Measuring the expression of the TUSC3 marker in a biological sample, preferably a tissue sample, and comparing it to the expression of the marker in biological samples from healthy women can contribute to diagnosing ovarian cancer. A corresponding method may comprise the following steps:
Isolating the RNA from the biological sample to be analyzed, preferably the tissue to be analyzed,
Producing cDNA by means of the isolated RNA, Performing a real time PCR for the TUSC3 marker,
Comparing the expression level of the marker from the biological sample to be analyzed to the level in the biological samples of healthy women.

TUSC3, originally named N33, was identified as a potential tumor suppressor gene at chromosome band 8p22 in prostate cancer around the mid-1990s (Bova G S, MacGrogan D, Levy A, Pin S S, Bookstein R, lsaacs W B. Physical mapping of chromosome 8p22 markers and their homozygous deletion in a metastatic prostate cancer. Genomics 1996; 35(1):46-54; MacGrogan D, Levy A, Bova G S, lsaacs W B, Bookstein R. Structure and methylation-associated silencing of a gene within a homozygously deleted region of human chromosome band 8p22. Genomics 1996; 35(1):55-65). The basis for this hypothesis was the identification of a plurality of homozygous deletions from the human chromosome band 8p22 in prostate cancer cells. The protein prediction showed that the coded protein is a presumed subunit of the oligosaccharyltransferase complex (Kelleher D J, Karaoglu D, Mandon E C, Gilmore R. Oligosaccharyltransferase isoforms that contain different catalytic STT3 subunits have distinct enzymatic properties. Molecular Cell 2003; 12(1):101-111). TUSC3 was homozygously deleted in several cancer cell lines, but rarely found in mutated form. Mutation analyses, which were conducted for prostate cancer and other types of tumors that frequently indicate loss of heterozygosity (LOH) and/or homozygous deletions at 8p22, showed no significant rate of deactivating mutations. Missense mutations were difficult to interpret due to the absence of known protein functions, and therefore interest in TUSC3 and the coded protein thereof dissipated.

Insights about haploinsufficiency and epigenetic modifications of tumor suppressor genes have rekindled the interest in old marker candidates.Databases and in-silico tools made available recently support these efforts.

Using publicly available gene expression data, the inventors systematically searched for the reduced expression of genes at 8p22 (unpublished data) and identified TUSC3 (N33) as one of the genes, the expression of which is significantly reduced in the presence of ovarian cancer. A quantitative expression analysis using real-time RT-PCR confirmed the dramatic decrease of TUSC3 in ovarian cancer tissues. The statistical analysis of the correlation of TUSC3 expression with clinical parameters showed that, at the time of the diagnosis, patients with reduced TUSC3 expression have overall a shorter survival period than patients whose TUSC3 levels in the tumors were identical to those in the normal ovarian tissues. These results were studied further and retested based on a large independent patient population. The initial observations of a potential prognostic significance of TUSC3 expression was confirmed.

The inventors of the present invention have found that TUSC3 can also serve as a molecular marker for the diagnosis of and/or making predictions about ovarian cancer, and can be used for assessing the efficacy of platinum-based chemotherapy and/or a therapy based on DNA-demethylating agents, such as decatibine (5-aza-2′-deoxycytidine) or zebularine, and/or based on inhibitors of histone deacetylases for ovarian cancer, in that the promoter hypermethylation of the TUSC3 marker in a biological sample, preferably in a tissue sample or a biological fluid, such as whole blood, serum or plasma, is measured: The method preferably comprises the following steps:
Isolating the DNA from the biological sample, preferably the tissue sample or biological fluid
Performing a PCR-based methylation assay
Analyzing the PCR products gained.

Advantageously, the PCR-based methylation assay is carried out with methylation-specific PCR. To this end, for the detection of methylated DNA, a PCR, for example with one of the following primer pairs, can be conducted:

5′-TTTTCGGTGAATCGGATG-3′ and
5′-TACGCGCCCAACTCCTA-3′,
5′-GGTTTAGTTAGCGGGTTTTCGG-3′ and
5′-AAACAAATACCGCAACCGCCG-3′ or
5′-GGTCGGGTAGGCGTGGTGCG-3′ and
5′-CCGCCCCGCTTACCTACGACGT-3′.

In addition, for the detection of non-methylated DNA, a PCR can be conducted with one of the following primer pairs:

5′-GGGTTTAGTTAGTGGGTTTTTGGAG-3′ and
5′-AAACAAATACCACAACCACCACCC-3′ or
5′-GAGGTTGGTTGGGTAGGTGTGGTGTG-3′ and
5′-CACAACCACCACCCCACTTACCTACAACAT-3′.

Alternatively, the PCR-based methylation assay can be carried with a methyl-sensitive restriction endonuclease reaction and PCR.

For this, for example the following method can be used:

For the specific detection of methylated sequences, 500 ng of a genomic DNA is mixed with 40 units of the restriction enzyme Hpa II (New England Biolabs, Ipswich, Mass., USA), using the reaction buffer NEBuffer 1 (New England Biolabs, Ipswich, Mass., USA), in a volume of 50 μl, covered with mineral oil, and treated at 37° C. for 18 hours. Then, the digested DNA is cleaned with the Invisorb Spin PCRapid Kit (Invitek, Berlin, Germany) and received in a volume of 50 μl. The PCR batch comprises 25 μl and is made of 0.3 μM each of the two biotinylated primers (forward: 5′-bio-GCCTTTCCAGGTCTTCTCC-3′ backward: 5′-bio-CCACGCGCGGTAGCCGTG-3′), 50 ng of the digested and cleaned DNA, 1 unit SuperTaq (HT Biotech, Cambridge, UK), 1× SuperTaq buffer (HT Biotech, Cambridge, UK), and 0.2 mM of the dNTPs (Larova, Teltow, Germany). The PCR is conducted under the following conditions: 2 min. at 94° C., 40 cycles—sec. at 94° C., 30 sec. at 58° C., 30 sec. at 72° C.—and finally 3 min. at 72° C. The detection of the PCR products that developed is performed with 2% agarose gel and ethidium bromide staining. In addition, the use of biotinylated primers also allows an analysis of the PCR products using alternative methods, for example sequence-specific capturing as part of reverse hybridization, and the subsequent detection of the bound biotinylated sequences using streptavidin enzyme conjugate.

The test kit according to the invention for the diagnosis and/or making predictions about ovarian cancer and/or for the assessment of the efficacy of platinum-based chemotherapy and/or a therapy based on DNA-demethylating agents and/or based on inhibitors of histone deacetylases for ovarian cancer comprises PCR primers that are suited for performing a methylation-specific PCR in the TUSC3 promoter region.

Alternatively, the test kit may comprise a restriction enzyme and PCR primers that flank the corresponding restriction interface within the TUSC3 promoter region.

In addition, the test kit may comprise means for detecting PCR products that developed, such as a hybridization probe.

With the method according to the invention for planning a regimen for the treatment of ovarian cancer, a method is performed for assessing the efficacy of platinum-based chemotherapy and/or a therapy based on DNA-demethylating agents, such as decitabine (5-aza-2′-deoxycytidine) or zebularine, and/or based on inhibitors of histone deacetylases for ovarian cancer, wherein the promoter hypermethylation of the TUSC3 marker is measured in a biological sample to be analyzed, preferably in a tissue sample to be analyzed or a biological fluid, and wherein, if hypermethylation of the TUSC3 promoter is detected, a therapy based on DMA-demethylating agents, such as decitabine (5-aza-2′-deoxycytidine) or zebularine, and/or based on inhibitors of histone deacetylases, is proposed.

With a method for planning a regimen for the treatment of ovarian cancer, a method is performed for assessing the efficacy of platinum-based chemotherapy and/or a therapy based on DNA-demethylating agents, such as decitabine (5-aza-2′-deoxycytidine) or zebularine, and/or based on inhibitors of histone deacetylases for ovarian cancer, in that the promoter hypermethylation of the TUSC3 marker is measured in a biological sample to be analyzed, preferably in a tissue sample to be analyzed or a biological fluid, and, if no hypermethylation of the TUSC3 promoter is detected, platinum-based chemotherapy is proposed.

The promoter hypermethylation of tumor suppressor genes (TSGs) as a molecular marker is very promising. It can provide insights into the biology of a tumor, and contrary to gene or protein expression, it is easy to detect using PCR-based methods not only in tumor material, but also in body fluids (Das P M, Singal R. DNA methylation and Cancer. J Clin Oncol 2004; 22(22):4632-4642; Jones P A, Baylin S B. The fundamental role of epigenetic events in Cancer. Nat Rev Genet 2002; 3(6):415-428; Laird P W. The power and the promise of DNA methylation markers. Nat Rev Cancer 2003; 3(4):253-266).

The hypermethylation of the chromosome band comprising TUSC3 was studied in prostate, colon, liver, lung and bladder cancer (MacGrogan D, Levy A, Bova G S, lsaacs W B, Bookstein R. Structure and methylation-associated silencing of a gene within a homozygously deleted region of human chromosome band 8p22. Genomics 1996; 35(1):55-65. Ahuja N, Li Q, Mohan A L, Baylin S B, Issa J P.

Aging and DNA methylation in colorectal mucosa and cancer. Cancer Res 1998; 58(23):5489-5494). Age-dependent hypermethylation was found only in normal and neoplastic colon tissue, but not with other types of cancer. Recently, another group found no hypermethylation of TUDC3 in 65 color cancer samples that were examined (Xu X L, Yu J, Zhang H Y et al Methylation profile of the promoter CpG islands of 31 genes that may contribute to colorectal carcinogenesis. World J Gastroenterol 2004; 10(23):3441-3454).

Hypermethylation was studied recently in a wide range of cancer-related genes in ovarian cancer. The selection of the genes was primarily random and based on the availability of established detection systems using methylation-specific PCR (Makarla P B, Saboorian M H, Ashfaq R et al Promoter hypermethylation profile of ovarian epithelial neoplasms. Clin Cancer Res 2005; 11(15):5365-5369; Teodoridis J M, Hall J, Marsh S et al CpG Island Methylation of DNA Damage Response Genes in Advanced Ovarian Cancer. Cancer Res 2005; 65(19):8961-8967). While these results are generally valuable for developing screening tests for cancer diseases, promoter hypermethylation can highlight relatively late and non-specific events in carcinogenesis. An ideal marker would also provide indications of the biological conduct. Recently, it was possible to identify p15^{INK4b} as a risk factor for ovarian cancer, after correction with respect to established prognostic factors in a multivariate Cox regression model (Liu Z, Wang L E, Wang L et al Methylation and Messenger RNA Expression of p15INK4b but Not p16INK4a Are Independent Risk Factors for Ovarian Cancer. Clin Cancer Res 2005; 11(13):4968-4976).

The inventors have found that the percentage of patients resistant to platinum-based chemotherapy was significantly higher in the group with hypermethylated TUSC3 promoters, which implies a certain necessary expression strength of TUSC3 for a favorable response to chemotherapy.

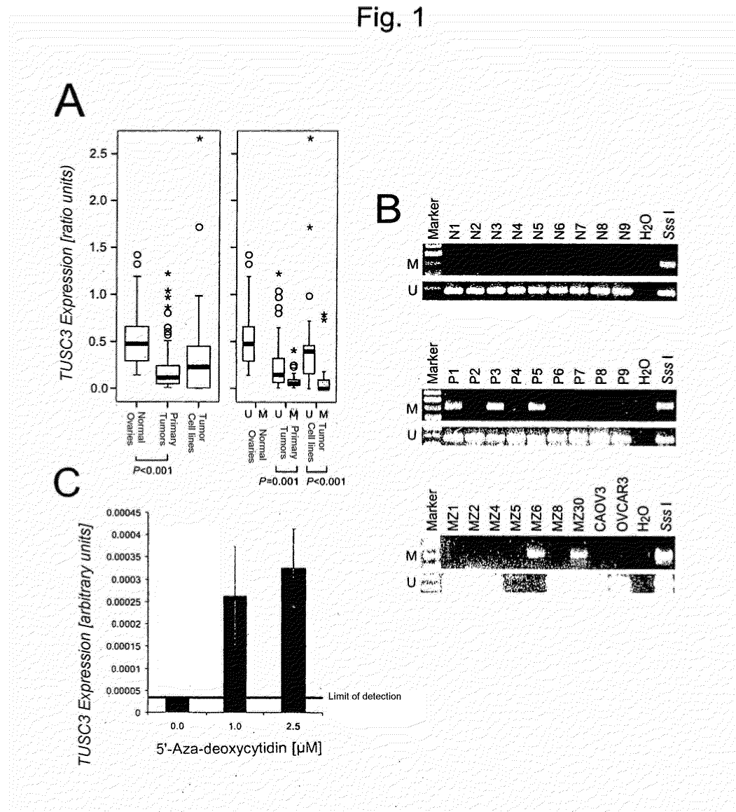

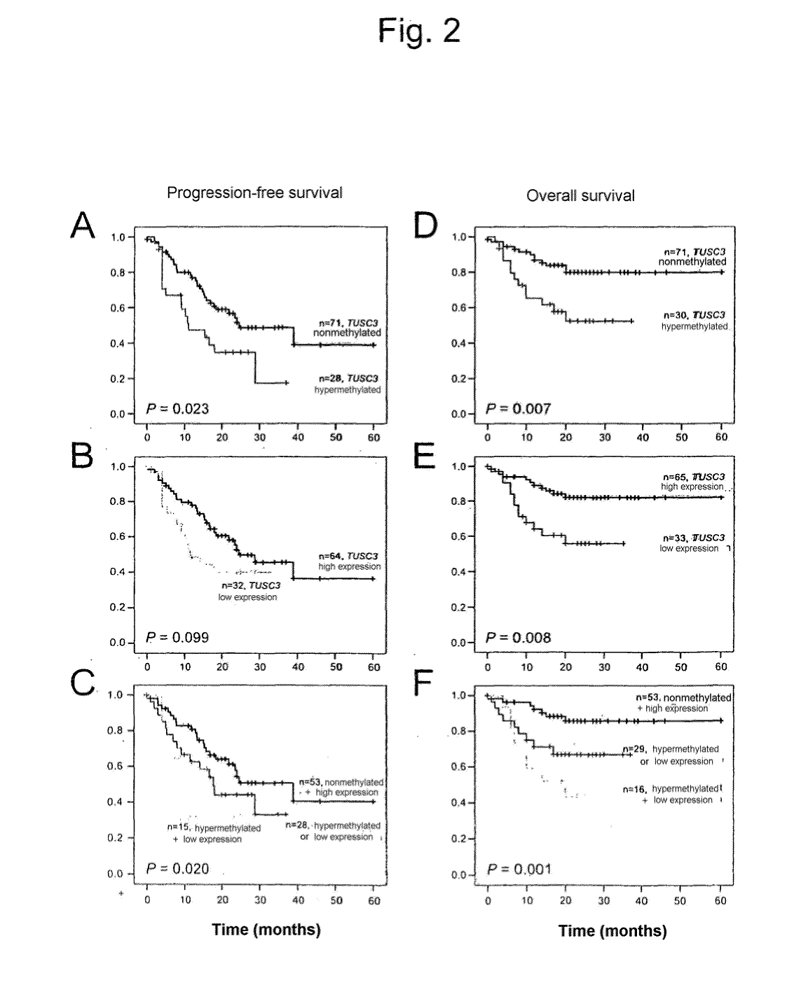

Below, studies that support the findings on which the present invention is based will be described. To this end, reference is also made to the enclosed drawing, in which FIG. 1A shows boxplots of the overall TUSC3 expression and the expression of the non-methylated and hypermethylated fractions of benign ovarian tissue samples, ovarian cancer tissue samples and ovarian cancer cell lines, FIG. 1B shows ethidium bromide agarose gel images of methylation-specific PCR products of benign ovarian tissue samples, ovarian cancer tissue samples and ovarian cancer cell lines, FIG. 1C shows the restoration of TUSC3 expression in ovarian cancer cell lines using 5-aza-deoxycytinde, and FIG. 2 shows Kaplan-Meier plots for progression-free and total survival of patients whose tumor samples exhibited different expression intensity levels and methylation states.

Studies

Patient Material and Cancer Cell Lines

Tumor samples were obtained from ovarian cancer patients at the time the primary diagnosis of ovarian cancer was made and who had undergone surgery. Normal ovarian samples enriched with epithelial cells and samples of benign cysts were derived from patients who were diagnosed without a malignant disease. In total, 20 benign ovarian samples and 102 primary tumor samples from 102 patients were analyzed.

As expected for a representative ovarian cancer population, based on the tumor samples the majority of patients (72%) was diagnosed with advanced stages (III and IV). 84% of the patients were treated with a platinum-based chemotherapy regimen. The corresponding data is summarized in Table 1.

TABLE 1
Clinical-pathological characteristics of ovarian cancer patients

| A) Benign ovarian samples (corresponding mRNA | 20 | (20) |
|---|---|---|
| samples) | | |
| Age during surgery [years] | | |
| Mean ± standard deviation | 49.9 ± 11.8 | |
| 7 normal ovaries, mean age [years]: | 51.6 | |
| 13 benign cysts, mean age [years]: | 49.0 | |
| B) Tumor samples (corresponding mRNA samples) | 102 | (99) |
| Age during diagnosis [years] | | |
| Mean ± standard deviation | 58.3 ± 11.0 | |
| Histology | | |
| Serous | 77 | (75.5% ∝) |
| Endometrioid | 10 | (9.8%) |
| Mucinous | 5 | (4.9%) |
| Clear-celled | 2 | (2.0%) |
| Other | 7 | (6.9%) |
| FIGO (2 (2.0%) missing) | | |
| I | 17 | (16.7%) |
| II | 10 | (9.8%) |
| III | 51 | (50.0%) |
| IV | 22 | (21.6%) |
| Grading (1 (1.0%) missing) | | |
| 1 | 2 | (2.0%) |
| 2 | 48 | (46.1%) |
| 3 | 51 | (50.0%) |
| Chemotherapy (3 (2.9%) missing) | | |
| Carboplatin and Taxol | 63 | (61.8%) |
| Carboplatin, taxol and gemcitabine | 17 | (16.7%) |
| Carboplatin or taxol monotherapy | 3 | (2.9%) |
| Carboplatin and other | 8 | (7.8%) |
| Other | 3 | (2.9%) |
| None | 5 | (4.9%) |
| Response to chemotherapy (16 (15.7%) missing) | | |
| pNED‡ | 6 | (5.9%) |
| pNC† | 3 | (2.9%) |
| pPD† | 12 | (11.8%) |
| pPR† | 5 | (4.9%) |
| pCR‡ | 43 | (42.2%) |
| Duration of response | | |
| 0-6 months, †, ‡ | 6 | (5.9%) |
| 6-12 months, ‡ | 5 | (4.9%) |
| >12 months ‡ | 6 | (5.9%) |
| Remissions/progressive diseases (3 (2.9%) missing) | | |
| Without remission | 49 | (48.0%) |
| With remission | 33 | (32.4%) |
| Progressive disease | 17 | (16.7%) |

∝ Percentage based on 102 tumor samples.
† Not responding to chemotherapy
‡ Responding to chemotherapy

In addition, 32 ovarian cancer cell lines were studied, which were started from ovarian cancer tissue samples of 28 patients and cultivated as described (Mobus V1 Gerharz C D, Press U, et al, Morphological, immunohistochemical and biochemical characterization of 6 newly established human ovarian carcinoma cell lines. Int J Cancer. 1992; 52:76-84).

DNA and RNA Isolation

Genomic DNA from about 15 mg of frozen tissue was isolated with the DNeasy Tissue Kit (Qiagen, Hilden, Germany) as described by the manufacturer. The DNA quantity was measured using the PicoGreen dsDNA Quantitation Kit (Molecular Probes, Inc., Eugene, Oreg., USA) and the DNA quality was evaluated on a random sample basis using 0.3% agarose gel electrophoresis. DNA from ovarian cancer cell lines was isolated using the FlexiGen DNA Kit (Qiagen, Hilden, Germany).

Total RNA from frozen tissues was extracted using the Agilent Total RNA Isolation Mini Kit (Agilent Technology, Palo Alto, Calif., USA) according to the manufacturer's information. Total RNA from cancer cell lines was isolated with the RNeasy Mini Kit (Qiagen, Hilden, Germany). The RNA quality and quantity were assessed by means of capillary electrophoresis using RNA Nano Chips (Lab-on-a-Chip, Agilent Technologies, Palo Alto, Calif., USA).

Methylation-Specific PCR (MSP) and DNA Demethylation

For bisulfite treatment, 1 μg genomic DNA was digested with the restriction enzymes Hind III and Xba I, precipitated, dissolved and denatured for 20 minutes in 0.35 M NaOH. Then, the DNA was treated in 8 cycles (2 hrs. 75° C. and 1 min. 95° C.) with 1.75 M sodium bisulfite, 5.23 M urea and 0.52 mM hydroquinone in a PCR cycler. Using the QIAEX II Gel Extraction Kit (Qiagen, Hilden, Germany), the DNA was cleaned and the reaction was ended with 0.32 M NaOH (20 min. 37° C.). Finally, the DNA was precipitated with 1 μl glycogen with NH₄ acetate EtOH as the carrier, rinsed with 70% EtOH and dissolved in 40 μl H₂O. For the methylation-specific reaction, the PCR was conducted with 25 ng DNA treated with bisulfite, primers published by Xu et al (Xu X L, Yu J, Zhang H Y et al Methylation profile of the promoter CpG islands of 31 genes that may contribute to colorectal carcinogenesis. World J Gastroenterol 2004; 10(23):3441-3454), AmpliTaq Gold (Applied Biosystems, Foster City, Calif., USA), 1× optimized buffer B (Invitrogen, Carlsbad, Calif., USA), under the following PCR conditions: 10 min. at 94° C., 50 cycles of 30 sec. each at 94° C.—30 sec. at 50° C.—20 plus 1 sec. per cycle at 72° C., and finally 7 min. at 72° C. For the detection of non-methylated DNA, the PCR was conducted with the following primers (sense: 5′-GAGGTTGGTTGGGTAGGTGTGGTGTG-3′ and antisense: 5′-CACAACCACCACCCCACTTACCTACAACAT-3′) and similar PCR conditions as described above (except: 1× optimized buffer F (Invitrogen, Carlsbad, Calif., USA) and 56° C. annealing temperature). The PCR products were analyzed on 3% agarose gel, stained with ethidium bromide. Bisulfite sequencing was performed directly with the PCR products that were cleaned with the QIAquick PCR Purification Kit (Qiagen, Hilden, Germany). DNA demethylation of the cancer cell lines was carried out by a 72-hour treatment with 1 and 2.5 μM 5-aza-deoxycytidine (Sigma-Aldrich, St. Louis, Mo., USA) in cell culture medium, and the relative TUSC3 expression was evaluated by real time RT-PCR as described below.

cdNA Synthesis and Quantitative Real-Time RT-PCR

cDNA was synthesized from 1 μg DNase I-digested total RNA using the DuraScript RT-PCR Kit (Sigma-Aldrich, St. Louis, Mo., USA) and a 1:1 mixture of the provided oligo-(dT)₂₃ and random nonamer oligo, substantially as described in the manual. TaqMan real-time PCR was carried out, and the relative expression (compared by using a calibrator, a cNDA mixture made of a plurality of cell lines) was calculated from the threshold cycles (Ct) using the GeneAmp 5700 SDS v1.3 (Applied Biosystems) as follows: 2̂—[(Ct_{gene} substances from duplicated probes—Ct_{gene} substances from duplicated calibrator)—(Ct_{B2M} substances from duplicated probes—Ct_{B2M} substances from duplicated calibrator)]—(and listed as arbitrary “ratio units”), wherein the following Assay-on-Demand™ probes were used: TUSC3, Hs00185147_m1 and beta 2 microglobulin, Hs99999907_m1 (Applied Biosystems, Foster City, Calif., USA).

Statistical Analysis

The continuous variables are shown as a mean value and standard deviation, the category variables as absolute and relative frequencies. In order to be able to compare the frequencies between two or more groups, a Fisher's Exact test or a Fisher-Freeman-Halton test was conducted.

If sufficient approximation of a normal distribution was found, the mean values and 95% confidence intervals for TUSC3 expression were calculated on a logarithmic scale and then transformed back into the original scale. In order to be able to compare the TUSC3 expression between two or more groups, a t-test or a one-way ANOVA was conducted, using the log-transformed expression as the independent variable. Equivalently, the potential influence of hypermethylation on TUSC3 expression using a t-test was analyzed on a logarithmic scale.

The potential influence of TUSC3 expression and of hypermethylation on the progression-free and overall survival is illustrated in the corresponding Kaplan-Meier plots and quantified with the use of Cox proportional hazard regression models. For this purpose, the expression was dichotomized at the 33.33 percentile since approximately 30% of the samples was hypermethylated and a strong correlation was observed between the hypermethylation state and expression; furthermore, a combination of this dichotomous variable and the hypermethylation status was determined at three levels (3=low expression and hypermethylated, 2=either low expression or hypermethylated, 1=high expression and not methylated) and considered in the Cox regression models as a linear factor; as a result, the corresponding hazard ratio can be applied to both comparisons of adjoining groups (3 vs. 2 and 2 vs.

1). The progression time used was the day on which the first relapse was documented, or the progression of the disease was first documented, starting with the time of the initial diagnosis.

Univariate Cox models were used in order to highlight the influence of known prognostic factors and the three potentially new prognostic factors described above. For each of the new factors, a multivariate Cox model, having known prognostic factors, was used as the adjusting variable.

P-values of <0.05 were considered statistically significant. All calculations were conducted with the use of SAS Software Version 9.1 (SAS Institute Inc., Cary, N.C., USA, 2001); graphical representations were prepared using SPSS Software Version 12.0 (SPSS Inc. Headquarters, Chicago, Ill., USA).

Results

TUSC3 mRNA Expression is Reduced with Ovarian Cancer

The quantitative real-time RT-PCR of 99 ovarian cancer tissues and 20 benign ovarian tissues was conducted. FIG. 1A shows the boxplots of the expression of TUSC3 in arbitrary units (ratio units) based on the expression of B-2-microglobulin as an internal reference (housekeeping gene control) of benign ovarian tissue samples (normal ovaries), ovarian cancer tissue samples (primary tumors) and ovarian cancer cell lines (tumor cell lines). In the right part of the graph, the expression of TUSC3 was divided into non-methylated (U) and hypermethylated (M) fractions. For the statistical analysis, a t-test on a logarithmic scale was used. Compared to controls (0.465, Cl 0.347-0.623 ratio units), the expression in tumors was significantly lower (P<0.001) (0.105, Cl 0.082-0.135 ratio units). Significant differences in TUSC3-expression between the tumor samples of patients from different age groups (<50 and >50), samples from different FIGO stages, and samples with varying differentiation levels (grading) were not found (Table 2A). Only the expression in non-serous tumors was significantly lower (P=0.037, not corrected for multiple tests) compared to serous tumors (Table 2A).

TABLE 2
Comparison of the clinical-pathological characteristics
with TUSC3 expression and hypermethylation
A) TUSC3 expression

| Clinical-pathological | Expression of | |
|---|---|---|
| characteristics | TUSC3 | Comparison (P) |
| Benign ovarian samples | | |
| Median | 0.465 | |
| CI | 0.347-0.623 | |
| Tumor samples (comparison | | <0.001^{†} |
| of benign ovarian samples) | | |
| Median | 0.105 | |
| CI | 0.082-0.135 | |
| Age | | |
| ≦50 | 0.085 | 0.299^{†} |
| >50 | 0.114 | |
| Histology | | |
| Serous | 0.121 | 0.037^{†} |
| Non-serous | 0.064 | |
| FIGO | | |
| I | 0.084 | 0.764^{‡} |
| II | 0.124 | |
| III | 0.104 | |
| IV | 0.126 | |
| Grading | | |
| 1 | 0.068 | 0.188^{‡} |
| 2 | 0.135 | |
| 3 | 0.086 | |

B) TUSC3 hypermethylation

| Clinical-pathological | Hypermethylation of | | |
|---|---|---|---|
| characteristics | TUSC3 | | Comparison (P)^{#} |
| Benign ovarian samples | 0/20 | (0%) | |
| Primary tumor samples | | | |
| Age | 30/102 | (29.4%) | 0.003 |
| ≦50 | | | 0.218 |
| >50 | 5/26 | (19.2%) | |
| Histology | 25/75 | (33.3%) | |
| Serous | | | 0.200 |
| Non-serous | 21/79 | (26.6%) | |
| FIGO | 9/22 | (40.9%) | |
| I | | | 0.948 |
| II | 5/17 | (29.4%) | |
| III | 2/10 | (20.0%) | |
| IV | 15/51 | (29.4%) | |
| Grading | 7/22 | (31.8%) | |
| 1 | | | 0.102 |
| 2 | 2/2 | (100%) | |
| 3 | 15/48 | (31.2%) | |
| | 13/51 | (25.5%) | |

^{‡}ANOVA (calculated after logarithmic transformation, mean and CI are listed in the original scale after back-transformation).
^{†}t-test (as calculated above)
^{#}Fisher's Exact test and Fisher-Freeman-Halton test, accordingly.

The TUSC3 promoter is hypermethylated, and TUSC3 expression can be restored by DNA-demethylation in ovarian cancer cell lines

The inventors have demonstrated that it was not possible to detect the expression of TUSC3 in 5 out of 38 ovarian cancer cell lines and that in three additional cell lines the expression was more than 10 times lower than the mean expression of all cell lines (not shown here). Using methylation-specific PCR (MSP), it was found that the promoter of TUSC3 was hypermethylated in seven of these eight cell lines with little or no TUSC3 expression. FIG. 1B shows, by way of example, ethidium bromide agarose gel images of methylation-specific PCR products of nine benign ovarian tissue samples (N1-N9), nine ovarian tumor samples (P1-P9) and nine ovarian cancer cell lines (MZ1-OVCAR3). An Sss I (CpG methylase)-methylated human DNA was used as the positive control and H₂O as the negative control. M identifies the methylated PCR product and U the non-methylated PCR product. The expression of TUSC3 was significantly (P<0.001) dependent on the hypermethylation state (FIG. 1A).

A concentration-dependent (1 and 2.5 μM) restoration of TUSC3 expression was achieved by a 72-hour treatment with the demethylated substance 5-aza-deoxycytidine in the hypermethylated MZ6 cell line. In the sham-treated samples, the TUSC3 expression was below the detection limit. With the use of 2.5 μM 5-aza-deoxycytidine, TUSC3 expression was at least 9.5 times greater. The experiments were conducted three times, and the mean values of the relative TUSC3 expression (based on the expression of B-2-microglobulin) were illustrated with standard deviations.

In order to further determine the methylation level and test the specificity of the MSP system used, the methylation status of individual CpG islands located closed to the alleged TUSC3 promoter was evaluated by the bisulfite sequencing of three hypermethylated cell lines and two controls. The sequences of the PCR products that were obtained showed that almost all CpGs in the PCR products of the three ovarian cancer cell lines had methylated more than 75%, but none of the CpGs of the two control had methylated more than 10% (data not shown).

TUSC3 Hypermethylation in Ovarian Tumor Samples

In a next step, the analysis of the hypermethylation status of the TUSC3 promoter was expanded by methylation-specific PCR to a list of 102 ovarian tumor samples that were derived from patients during the primary diagnosis (FIG. 1B). Hypermethylation of TUSC3 was found in 29.8% (30/102) of these ovarian cancer cases, but in none of the 20 controls (P=0.003).

TUSC3 Hypermethylation Closely Correlates with TUSC3 Expression

FIG. 1 shows the TUSC3 expression in the hypermethylated and non-hypermethylated ovarian tumor tissues. The mean expression of TUSC3 in the hypermethylated group was significantly lower (0.065, Cl 0.038-0.081 ratio units) than in the non-methylated group (0.135, CI 0.100-0.182 ratio units, P=0.001), which confirms a similar interdependency of the TUSC3 hypermethylation status and TUSC3 expression, as that which was demonstrated above for ovarian cancer cell lines, and suggests a causal correlation between the hypermethylation status and the expression in primary tumor samples (FIG. 1A and Table 3).

TABLE 3
Significant differences of clinical-pathological
characteristics of hypermethylated tumor samples

| | TUSC3 Hypermethylation | | |
|---|---|---|---|
| Clinical-pathological | non- | | |
| characteristics | methylated | methylated | Comparison (P) |
| Age | n = 72 | n = 30 | 0.023^{†} |
| Mean ± standard | 56.7 ± 10.8 | 62.1 ± 10.7 | |
| deviations | | | |
| Range | 35-81 | 41-86 | |
| Expression of TUSCE3 | n = 71 | n = 28 | 0.001^{‡} |
| (qRT-PCR) | | | |
| Mean | 0.135 | 0.056 | |
| CI | 0.100-0.182 | 0.038-0.081 | |

^{†}t-test
^{‡}t-test (calculated after logarithmic transformation, mean and CI are stated after transformation back into original scale)

TUSC3 Hypermethylation and Clinical Characteristics

There was no significant difference in the hypermethylation frequency between the tumor samples from patients of different age groups (<50 and >50), samples with different histological characterization (serous vs. non-serous), samples from different FIGO stages, and samples from different differentiation levels (grading) (see Table 2B). It was interesting that the mean age in the hypermethylated group upon diagnosis was 5.4 years higher (P=0.023) than in the non-methylated group (Table 3). In the group of the 30 patients with hypermethylated TUSC3 promoters, a larger number of patients suffered from a progressive disease (32.1%) than in the group of the 72 patients without TUSC3 hypermethylation (11.3%), which suggests the potential influence of TUSC3 hypermethylation and expression on the prognosis of patients with ovarian cancer.

Correlation of the TUSC3 Hypermethylation Status with Progression-Free and Overall Survival

A significant correlation was found between TUSC3 hypermethylation and the progression-free survival (relative risk of 1.972, P=0.023) and overall survival (relative risk of 2.915, P=0.007) (FIGS. 2A and 2B, Table 4). The hazard ratios for progression-free survival were comparable to the FIGO stage (relative risk of 2.821, P<0.001) and the remaining tumor after surgery (relative risk of 2.867, P<0.001), the only other significant prognostic factors available at the time when options for systemic therapy have to be explored.

The mean progression-free survival was 11.10 (Cl 2.58-19.64) months in the hypermethylated group compared to 24.55 (Cl 11.55-37.55) months in the non-methylated group (FIG. 2a).

The correlation between TUSC3 hypermethylation and the prognosis of the patients was independent from other risk factors, including how the patients responded to systemic chemotherapy (interaction term not significant) for progression-free survival (relative risk of 2.024, P=0.042) and for overall survival (relative risk of 3.817, P=0.013) (Table 4).

The Kaplan-Meier estimates and hazard ratios that were obtained with the dichotomized TUSC3 expression levels, were similar to those obtained using the TUSC3 hypermethylation status (FIGS. 2B and 2E, Table 4). In cases where the hypermethylation status and expression level agreed, the influence on the progression-free and overall survival was even more significant (FIGS. 2C and 2F, Table 4).

TABLE 4
Univariate and multivariate Cox hazard ratio analysis

| Univariate | | Multivariate | |
|---|---|---|---|
| Relative risk ‡ | | Relative risk ‡ | |
| (95% CI) | P | (95% CI) | P |

Progression-free survival

| | | | | |
|---|---|---|---|---|
| Age upon diagnosis | 1.018 (0.992-1.045) | 0.184 | | |
| Histology (non-serous vs serous) ^{†} | 1.156 (0.576-2.323) | 0.683 | | |
| FIGO stage | 2.821 (1.867-4.260) | <0.001 | | |
| Grading | 0.978 (0.581-1.646) | 0.933 | | |
| Remaining tumor (yes vs no) ^{†} | 2.867 (1.615-5.091) | <0.001 | | |
| Response to therapy (no vs yes) ^{†} | 4.907 (2.644-9.104) | <0.001 | | |
| Methylation of TUSC3 (yes vs no) ^{†} | 1.972 (1.099-3.533) | 0.023 | 2.024 (1.025-4.000) | 0.042 |
| Expression of TUSC3 (low vs high) ^{††} | 1.661 (0.908-3.304) | 0.099 | 1.610 (0.726-3.571) | 0.242 |
| ^{L} combined (methylation and expression) ^{±} | 1.565 (1.072-2.283) | 0.020 | 1.510 (0.915-2.494) | 0.107 |

Total survival

| | | | | |
|---|---|---|---|---|
| Age upon diagnosis | 1.056 (1.018-1.095) | 0.003 | | |
| Histology (non-serous vs serous)^{†} | 2.369 (1.051-5.319) | 0.037 | | |
| FIGO stage | 2.518 (1.411-4.493) | 0.002 | | |
| Grading | 0.762 (0.378-1.534) | 0.446 | | |
| Remaining tumor (yes vs no) ^{†} | 5.278 (2.193-12.702) | <0.001 | | |
| Response to therapy (no vs yes) ^{†} | 9.560 (3.562-25.654) | <0.001 | | |
| Methylation of TUSC3 (yes vs no) ^{†} | 2.915 (1.348-6.289) | 0.007 | 3.817 (1.326-10.989) | 0.013 |
| Expression of TUSC3 (low vs high) ^{††} | 2.985 (1.333-6.711) | 0.008 | 2.079 (0.642-6.757) | 0.222 |
| ^{L} combined (methylation and expression) ^{±} | 2.267 (1.390-3.690) | 0.001 | 2.150 (01.047-4.405) | 0.037 |

‡ CI, confidence range.
^{†} Category variable.
^{††} Category variable (intersecting point 33.33 percentile)
^{±} Methylated and low expression vs. methylated and low expression vs. non-methylated and high expression.

1. A method for diagnosing or prognosticating ovarian cancer wherein the promoter hypermethylation of the TUSC3 marker is measured in a biological sample to be analyzed, preferably in a tissue sample to be analyzed or a biological fluid.

2. A method for assessing the efficacy of platinum-based chemotherapy or a therapy based on DNA-demethylating agents, such as decitabine (5-aza-2′-deoxycytidine) or zebularine, or based on inhibitors of histone deacetylases for ovarian cancer wherein the promoter hypermethylation of the TUSC3 marker is measured in a biological sample to be analyzed, preferably in a tissue sample to be analyzed or a biological fluid.

3. The method according to claim 1, further comprising the following steps:
isolating the DNA from the biological sample, preferably the tissue sample or biological fluid,
performing a PCR-based methylation assay, and
analyzing the PCR products gained.

4. The method according to claim 3 wherein the PCR-based methylation assay is performed with methylation-specific PCR.

5. The method according to claim 4 wherein during the methylation-specific PCR for the detection of methylated DNA a PCR is performed with one of the following primer pairs:
5′-TTTTCGGTGAATCGGATG-3′ and
5′-TACGCGCCCAACTCCTA-3′,
5′-GGTTTAGTTAGCGGGTTTTCGG-3′ and
5′-AAACAAATACCGCAACCGCCG-3′ or
5′-GGTCGGGTAGGCGTGGTGCG-3′ and
5′-CCGCCCCGCTTACCTACGACGT-3′.

6. The method according to claim 5 wherein, for the detection of non-methylated DNA, additionally a PCR is performed with one of the following primer pairs:
5′-GGGTTTAGTTAGTGGGTTTTTGGAG-3′ and
5′-AAACAAATACCACAACCACCACCC-3′ or
5′-GAGGTTGGTTGGGTAGGTGTGGTGTG-3′ and
5′-CACAACCACCACCCCACTTACCTACAACAT-3′.

7. The method according to claim 3 wherein the PCR-based methylation assay is performed with methylation-sensitive restriction endonuclease reaction and PCR.

8. A test kit for the diagnosis of or making predictions about ovarian cancer or for the assessment of the efficacy of platinum-based chemotherapy or a therapy based on DNA-demethylating agents or based on inhibitors of histone deacetylases for ovarian cancer, the test kit comprising PCR primers that are suited for performing a methylation-specific PCR in the TUSC3 promoter region.

9. A test kit for the diagnosis of or making predictions about ovarian cancer or for the assessment of the efficacy of platinum-based chemotherapy or a therapy based on DNA-demethylating agents or based on inhibitors of histone deacetylases for ovarian cancer, the test kit comprising a restriction enzyme and PCR primers that flank the corresponding restriction interface in the TUSC3 promoter region.

10. The test kit according to claim 8 wherein the test kit additionally comprises means for detecting PCR products that developed, such as a hybridization probe.

11. A method for planning the regimen for the treatment of ovarian cancer wherein the method for assessing the efficacy of platinum-based chemotherapy or a therapy based on DNA-demethylating agents, such as decitabine (5-aza-2′-deoxycytidine) or zebularine, or based on inhibitors of histone deacetylases for ovarian cancer is performed according to claim 2 and that upon the determination of hypermethylation of the TUSC3 promoter a therapy based on DNA-demethylating agents, such as decatibine (5-aza-2′-deoxycytinde) or zebularine, or based on inhibitors of histone deacetylases is proposed.

12. A method for planning the regimen for the treatment of ovarian cancer wherein the method for assessing the efficacy of platinum-based chemotherapy or a therapy based on DNA-demethylating agents, such as decitabine (5-aza-2′-deoxycytidine) or zebularine, or based on inhibitors of histone deacetylases for ovarian cancer is performed according to claim 2 and that platinum-based chemotherapy is proposed if no hypermethylation of the TUSC3 promoter is determined.


SEQUENCE LISTING (12 sequences)

SEQ ID NO: 1 - DNA, 18 bp, Homo sapiens
ttttcggtgaatcggatg

SEQ ID NO: 2 - DNA, 17 bp, Homo sapiens
tacgcgcccaactccta

SEQ ID NO: 3 - DNA, 22 bp, Homo sapiens
ggtttagttagcgggttttcgg

SEQ ID NO: 4 - DNA, 21 bp, Homo sapiens
aaacaaataccgcaaccgccg

SEQ ID NO: 5 - DNA, 20 bp, Homo sapiens
ggtcgggtaggcgtggtgcg

SEQ ID NO: 6 - DNA, 22 bp, Homo sapiens
ccgccccgcttacctacgacgt

SEQ ID NO: 7 - DNA, 19 bp, Homo sapiens
gcctttccaggtcttctcc

SEQ ID NO: 8 - DNA, 18 bp, Homo sapiens
ccacgcgcggtagccgtg

SEQ ID NO: 9 - DNA, 25 bp, Homo sapiens
gggtttagttagtgggtttttggag

SEQ ID NO: 10 - DNA, 24 bp, Homo sapiens
aaacaaataccacaaccaccaccc

SEQ ID NO: 11 - DNA, 26 bp, Homo sapiens
gaggttggttgggtaggtgtggtgtg

SEQ ID NO: 12 - DNA, 30 bp, Homo sapiens
cacaaccaccaccccacttacctacaacat